(19) United States
(12) Patent Application Publication
(10) Pub. No.: US 2020/0022905 A1
(43) Pub. Date: Jan. 23, 2020

(54) Composition for buccal absorption of nicotine for the purpose of smoking cessation and method for administering

(71) Applicant: HEGLUND, A.S., Praha (CZ)

(72) Inventors: Dobromil KO{hacek over (S)}ÍK, Praha (CZ); Milan Dittrich, Hradec Kralove (CZ)

(73) Assignee: HEGLUND, A.S., Praha (CZ)

(21) Appl. No.: 16/563,824

(22) Filed: Sep. 7, 2019

Related U.S. Application Data
(63) Continuation of application No. 13/147,597, filed on Aug. 3, 2011.

(30) Foreign Application Priority Data
Feb. 11, 2009 (CZ) PV 2009-80
Dec. 9, 2009 (CZ) PV 2009-829

(51) Int. Cl. A61K 9/00

(52) U.S. Cl. CPC A61K 9/006

(57) ABSTRACT

Composition intended for buccal absorption of nicotine into the system circulation and distribution to the central nervous system for the purpose of smoking cessation or nicotine substitution in conditions adverse for smoking according to the invention contains nicotine solution in form of a base and/or its salt with organic acid in concentration 0.01 to 8.00% by weight and substances with mucolytic effect. Single application dosage contains 0.05 to 3.00 mg of nicotine. A method for administering is also provided.

The present application is a continuation of U.S. application Ser. No. 13/147,597, filed Aug. 3, 2011, which is the U.S. national stage of International Application PCT/CZ2010/000011, filed Feb. 3, 2010, which claims priority to CZ Applications PV 2009-80, filed Feb. 11, 2009, and PV 2009-829, filed Feb. 19, 2009, all of which are incorporated by reference.

### TECHNOLOGY FIELD

The invention deals with a composition intended for buccal absorption of nicotine into the systemic circulation and distribution to the central nervous system for the purpose of smoking cessation or nicotine substitution in conditions adverse for smoking.

### EXISTING CONDITIONS OF TECHNOLOGY

Harmful effects of smoking have been covered by multiple extensive, statistically assessed studies carried out on large sets of smokers and non-smokers. By smoking of one cigarette pack per day smoker delivers to his/her system circulation a daily dose of 20 to 30 mg (Benowitz N L et al., Clin. Pharmacol. Ther. 44 (1988) 23-28). Nicotine absorbs very quickly, after smoke inhalation it gets to the brain during ten to twenty seconds (Benowitz N L et al., Annu. Rev. Pharmacol. Toxicol. 36 (1996) 597-613). Many proofs exist that brain creates tolerance and addiction to nicotine after its effect on dopaminergic receptors in the brain (Hoffman D et al., Am. J. Health 73 (1983) 1050-1053). It was clearly proved that smoking cessation involves dishabituation to nicotine addiction; therefore it is a nicotinism therapy. It is a usually a long-term and relatively poorly successful process (Cummings K M, Hyland A, Ann. Rev. Pub. Health 26 (2005) 583-599).

Smoking cessation therapies use preparations with various types of nicotine applied in various ways. In order to facilitate application and achieve sufficient nicotine bioavailability the most common absorption method is via buccal mucosa. Smoker endeavours to achieve plasmatic concentration of nicotine within the range from 30 to 50 μg/l (Lawson G M et al., J. Clin. Pharmacol. 38 (1998) 510-516). Slow release of nicotine is not advantageous, key aspect of the addiction is pharmacological response to instantly achieved plasmatic peak of nicotine (Henningfield J E, Keenan R M, J. Consult. Clin. Psychol. 61 (1993) 743-750).

Bioavailability of chewing gums Shiffman S et al., Addiction 97 (2002) 505-516) containing 2 mg or 4 mg of nicotine is approximately 50% (Benowitz N L, Jacob P III, Savanapridi C, Clin. Pharmacol. Ther. 41 (1987) 467-473). Plasmatic profiles of nicotine concentration over time vary from those during smoking in stability of its levels due to slow and continuous release of nicotine into oral cavity (Henningfield J E et al., Drug Alcohol Depend. 33 (1993) 23-29). Nicotine bioavailability to a large extent depends on pH value in the mouth, no too acid drinks are recommended 15 minutes before and after application of chewing gum (Henningfieid J E et al., JAMA 264 (1990) 1560-1564) because salts of in its ionized form feature decreased transmucosal absorption. This application form also causes unpleasant hot sensation in mouth and throat (Henningfield J E et al., CA Cancer J. Clin. 55 (2005) 281-299). Nicotine release rate is rather non-reproducive, some producers recommend not to chew too intensively to avoid overdosing (U.S. Pat. No. 6,344,222).

Lozenges are similar form of application as chewing gums, nicotine is released by slow dissolving in mouth (U.S. Pat. No. 4,967,773), much slower than during smoking (Russel M A H et al., Lancet 2 (1985) 1370). Composition with more rapid dissolution of nicotine is patented (U.S. Pat. No. 6,280,761). To achieve higher bioavailability, pH values from 7 to 9 were recommended, which are controlled by carbonate buffer; composition was sufficiently stable (U.S. Pat. No. 6,280,761). By increasing the pH value from 7.4 to 8.5 higher ratio of non-ionized form was achieved from 30% to 80% and bioavailability increased 3.8 times (U.S. Pat. No. 6,110,495). Low efficiency of lozenges was attributed to low nicotine dosage. Therefore in the USA, higher dosages were permitted from 1 mg to 2 mg and 4 mg (Shiffman S et al., Arch. Intern. Med. 162 (2002) 1267-1276), Nicotine release is slow, bioavailability was proved by 25% higher than with chewing gums (Choi J H et al., Nicotine Tob. Res. 5 (2003) 635-644), Another, less common application form, is sublingual tablets. These are very small tablets soluble in water to be placed under the tongue. Nicotine bioavailability in one dosage is similar as with chewing gum (Molander L, Lunell E, Eur. J. Clin. Pharmacol, 56 (2001) 813-819).

Nasal sprays are, another application form of nicotine. Preparations of this kind are in the USA available only upon medical prescription (Henningfield J E et al., CA Cancer J. Clin. 55 (2005) 281-299). Dosages by 0.5 mg of nicotine in 50 μl of solvent are applied to each naris. Nicotine is absorbed very rapidly, their advantage is individual dosing of nicotine according to patient's addiction level Schneider N G et al., Clin. Pharmacokinet. 31 (1996) 65-80). First dosages of this preparation very often induce nasal mucous membrane irritation.

Buccal spray, based on alcohol, water or macrogol solution of active substances and flavouring additives with focus on nicotine and its salts, clemastin and steroid hormones sprayed by a pump, is subject to patents WO/1997/038662 and EP 0 904 055. Use of a pump spray for nicotine application into oral cavity has also been described (U.S. Pat. No. 5,186,925); arrangement of oral spray with nicotine incorporated into microspheres is patent protected as well (U.S. Pat. No. 5,939,100).

Novel and at present favourite type of products for nicotine application is a so-called electronic cigarette (Pat. EP Appl. 1618803). Nicotine is absorbed by oral cavity and lungs after inhalation. The World Health Organization issued an official statement that they do not recommend this product as an aid to smoking cessation due to lack of information on its safety and efficiency (Anon., Marketers of electronic cigarettes should halt unproved therapy claims. World Health Organization, 2008-09-19).

Rather problematic may be the influence of not negligible concentration of exhaled nicotine vapours in constrained spaces in transport vehicles on health of non-smokers and especially children whose nicotine detoxifying mechanisms are considerably less efficient than adults'.

Nicotine is a very efficient natural substance belonging to group of alkaloids. It features very rapid start of effect, it influences multiple functions, inter glia heart, blood-vessel and brain. Lethal dose (LD 50) is between 30 and 60 mg (Gosselin R E, Clinical toxicology of commercial products, VI. Ed., Williams & Wilkins, Baltimore 1988, pp 311-313), serious toxic reactions may be experienced, especially by non-smokers, already after dose of 4 to 8 mg, with children this dose is even lower. No antidote efficient against this substance is known.

Transfer of all alkaloids, including nicotine, through biological barriers is easier when their solutions are applied in non-ionized form, i.e. at levels of current acidity exceeding as much as possible the pka value. The pka value is a parameter, which expresses the pH value, at which the concentrations of ionized and non-ionized forms of a substance are equal. When applying biologically active substances it is also necessary to consider physiological conditions. Certain compromise between physiologically optimal pH value (isoacid solution) and pH value optimal for permeability through barriers is a so-called euacid solution. This euacid pH value guarantees sufficient bioavailability at pH value suitable also for biological environment as well as for applied substance. Biological availability is parameter, which expresses ratio from the total amount of applied substance, which gets into system circulation or to the target tissue or structure.

In case of many substances the euacid pH value is achieved by combining suitable ratio of a substance in form of the acid or base and its salt. In case of nicotine according to the invention it involves a combination of nicotine base with its salt formed by addition of suitable amount of acid.

In addition to increasing the ratio of non-ionized form as a means to increase the substances bioavailability, specific option in case of buccal application of substances is to decrease barrier function of mucin. Mucin is a high-molecular glycoprotein featuring high viscosity of water solutions. Its viscosity can be reduced by substances called mucolytic agents. From today's perspective, already a classic group of mucolytic agents is compounds containing in their molecule a sulfhydryl group, which causes reduction of cross-linking of mucin molecule by mechanism of reduction of disulphide groups. Reaction is accompanied by significant decrease of solution viscosity, which is exploited in the sphere of solving symptoms of pulmonary diseases and respiratory system diseases of various seriousness. This is the effect of amino acids cysteine, methionine and their esters and ethers (Takatsuka S et al., Int. J. Pharm. 349 (2008) 94-100).

There are many other, non-sulphydryl group based mucolytic agents, acting by various mechanisms. Various enzymes have depolymerization effect on mucin (Rubin B K, Respiratory Care, 52 (2007) 859-865). Traditionally used in practice are solutions of electrolytes, such as ammonium chloride and sodium chloride, causing the mucin's macromolecules covering layer to dehydrate partially, thus decreasing viscosity of its solution. Use of processed sea water as a mucolytic agent in aerosol form is patent protected (U.S. Pat. No. 7,097,852). Combinations of electrolytes with hypertonic solutions of saccharides (honey, figs, etc.) or polyhydric alcohols (mannitol, sorbitol, xylitol, pentaerythritol) are used in traditional and alternative medicine mainly in paediatrics.

In therapeutic practice, many phytocomplexes obtained by extraction from various parts of plant are widely used. Sustained tradition attained extractions from blossoms or roots of plant Primula veris or Primula elatior containing o triterpene saponines and phenolic glycosides (Anon.: Assessment report on Primula veris, Primula elatior (L), radix. EMEA(HPMC/144474/2006), Saponins have significant effect in water solution by surface activity mechanism. Use of plant saponins and sapogenins for decreasing the human sputum viscosity as part of its microbiological analysis is patent protected (U.S. Pat. No. 4,053,363). Similar effect is shown by triterpene saponins contained in leaves of Hedera helix (Kraft K, Zeitschr. Phytotherap. 25 (2004) 179-181; Fazio S et al., Phytomedicine 16 (2009) 17-24). Essential oils of Eucalyptus globulus contain mainly 1,8-cineol (eucalyptol) and also various substances, such as terpenes, sesquiterpenes and sesquiterpenois acting using various mechanisms, inter glia also mucolytic one (Tibballs J, Med. J. Aust. 163 (1995) 177-180). Also roots of Liguiritia glabra offer a complex of surface active substances, which after separation of undesired glycyrrhizin has mucolytic effects (Guslandi M, Int. J. Clin. Parmacol. Ther. Toxicol., 23 (1985) 398-402). Combination of substances contained in Lichen islandicus and Althea officinalis together with local anaesthetic is part of WO12006/032364, Blossoms of species Tussilago farfara and genus Verbascum contain a complex of substances, in particular flavonoids and saponins types, which inter alia have also mucolytic effect (Newell C A, Anderson L A, Phillipson J D, Herbal Medicines, Pharmaceutical Press, London 1996). There are many other phytocomplexes used in practice with declared mucolytic effect, such as Euphrasia, Origanum, Pimpinella, Thymus, Trifolium, etc.

Nicotine is a relatively stable substance; exposed to air it decomposes by oxidation, decomposition is accelerated by light. Reaction is catalyzed by strong acceptors of electrons, inhibited by substances capturing radicals. During decomposition reaction are produced mainly N-oxides of alkylpyrrolidines (Suffredini H B et al., Anal. Letters 38 (2005) 1587-1599) and in smaller extent also pyrrolidinones (Beckwith A U et al., Austral. J. Chem. 36 (1983) 719-739). In nicotine adsorbed on various celluloses was found also kotinin, methanon-(1-methyl-3-pyrrolidinyi)-3-pyridinyl, and other degradation products (Mihranyan Andersson S-B Ek R Eur. J. Pharm. Sci. 22(2004) 279-286). One of the methods to stabilize nicotine is its adsorption on cellulose from algae, bacteria or fungi (WO/2005/023227). Nicotine is metabolized especially in liver, tens of metabolites were found (Moyer T P et al., Clin. Chem. 48 (2002) 1460-1471). In order to stabilize nicotine addition of water soluble antioxidants is necessary.

### SUMMARY OF THE INVENTION

Composition intended for buccal absorption of nicotine into the system circulation and distribution to the central nervous system for the purpose of smoking cessation or nicotine substitution in conditions adverse for smoking according to the invention is characterized by the fact that it contains nicotine solution in form of a base and/or its salt with organic acid in concentration 0.01 to 8.00% by weight, more preferably 0.10 to 4,00% by weight, most preferably 0.20 to 1.00% by weight, further it contains substances with mucolytic effect selected from the group formed by acetylcysteine in the amount of 3.0 to 30.0% by weight and/or phytocomplexes with mucolytic effect obtained by extraction from plants selected from the group of Primula veris, Primula elatior, Hedera helix, Eucalyptus globulus or Liquiritia glabra in concentration from 0.50 to 15.00% by weight.

Composition may further contain 0.05 to 1.50% by weight of phytocomplexes with antioxidizing effect selected from the group of flavones, flavonols, flavanones, flavanonols, isoflavones, neoflavonoids, anthocyanidins, proanthocyanidins and their oligomers and polymers obtained by extraction from plants Camellia sinensis, Curcuma longa, Rosmarinus officinalis, Sylibium marianum, Vitis vinifera, Ginkgo biloba, Euterpe oleracea, Vaccinium myrtilius, Morinda citrifolia, Malpighia glabra.

Composition advantageously further contains buffer or substance adjusting current acidity in the range of pH value from 6.0 to 9.5, more preferably from 7.0 to 9.0, most preferably 8.5.

As a solvent, the composition contains water in concentration 50 to 99% by weight or its mixture with polyhydric alcohol, such as glycerol, propylene glycol, and/or saccharide selected from the group of mannitol, xylitol, glucose, fructose, saccharose in concentration from 2 to 50% by weight.

Composition also advantageouslycontains at least one component from the group of substances adjusting taste perceptions, substances adjusting olfactory perceptions, antimicrobials, sequestrants, antioxidants, solubilizing agents and agents facilitating wettability, substances improving physical parameters of applied compositions, such as viscosity and surface tension, and substances improving condition of oral cavity surfaces, i.e. mucosae or teeth.

Composition according to the invention is intended for application of nicotine into oral cavity in form of colloid or heterogene aerodispersion by spray device with dispenser with individual dosages volume in the range from 50 μl to 2000 μl, preferably from 150 μl to 500 μl.

Single application dose contains 0.05 to 3.00 mg, more preferably 0.10 mg to 2.00 mg, most preferably 0.25 mg to 1.00 mg of nicotine.

### DETAILED DESCRIPTION OF THE PREFERRED EMBODIMENTS

Apart from nicotine and its salt as an active substance, the composition contains mucolytic agent, such as acetylcysteine and/or phytocomplexes with mucolytic effect obtained by extraction from plants as a nicotine bioavailability enhancer. It may further contain antioxidant of natural plant origin as a substance stabilizing the nicotine against oxidation and water as a solvent.

Other ingredients, which may be advantageously exploited in the composition, are stabilizers of various type, i.e. antimicrobials, sequestrants, free radical quenching agents; substances adjusting organoleptic properties may also be used. Compositions according to the invention may also include substances, improving physical parameters of applied compositions, such as viscosity and surface tension, and substances improving conditions of oral cavity surfaces, i.e. mucosae or teeth.

Nicotine is very efficient alkaloid readily soluble in water to strongly alkaline solution. As its tolerance by the buccal mucosa is good and absorption through buccal mucosa into the system circulation is sufficient, it is suitable that pH value of nicotine solutions is lower than 9.0 and higher than 7.0. It is convenient to reduce the pH value of water solutions of nicotine using their mixture with salts of acids, such as citric acid, malic acid, tartaric acid, lactic acid, glycolic acid, phosphoric acid, etc. Concentration of nicotine in solution is determined by volume of applied dosages; it can be from 0.01% to 8.00%, more preferably from 0.10% to 4.00%, most preferably from 0.20% to 1.00%. Individual applied dosages of nicotine may be in the range from 0.05 mg to 3.00 mg, more preferably from 0.10 mg to 2.00 mg, most preferably from 0.25 mg to 1.00 mg.

Mucolytic agents are strongly hydrophilic substances reducing viscosity of mucin, such as substances of mucoprotein type. Reduced viscosity of mucoproteins relates to faster diffusion of nicotine molecules to the surface of mucosa barrier, thus leading to faster and more intense absorption of this substance. Acetylcysteine is a strongly hydrophilic substance. Carboxyl group may interact with basic nitrogen of pyrrolidine cycle of nicotine. It has antioxidizing effect, sulfhydryl group may be a donor of electrons. It is a significant mucolytic agent, it reduces viscosity of water solutions of mucoproteins by interaction with disulphide bonds.

Mucolytic agents of natural plant origin suitable for increase of nicotine bioavailability in the composition are identical with those traditionally used as expectorant substances facilitating expulsion of sputum, especially in paediatrics. Phytocomplexes are therefore used, obtained by extraction from plant drugs, such as water or alcohol extracts from blossoms or roots of plant Primula veris or Primula elatior containing triterpene saponins and phenolic glycosides. Triterpene saponins contained in leaves of Hedera helix have similar effects. Composition according to the invention also may suitably contain essential oil obtained from leaves of Eucalyptus globulus, which contains especially 1,8-cineol (eucalyptol) and also various substances such as terpenes, sesquiterpenes and sesquiterpenols. Also phytocomplex from Liquiritia glabra obtained by extraction from roots offers surface active substances, which after separation of undesired glycyrrhizin have mucolytic effects. Another suitable ingredient in the composition is a combination of substances contained in Lichen islandicus, Althea officinalis, and also substances contained in species Tussilago farfara and genus Verbascum. There are many other phytocomplexes, which can be used with required mucolytic effect, such as Euphrasia, Origanum, Pimpinella, Thymus, Trifolium, etc. Phytocomplexes with mucolytic effect may be used as an ingredient of the composition in concentration from 0.5% by weight to 15% by weight, more preferably in concentration from 0.5% by weight to 5% by weight, most preferably in concentration from 1% by weight to 2% by weight.

Antioxidants are substances, which in given composition are significant for nicotine stabilization in its solution against oxidizing reactions, also they have positive effect on buccal mucosa. As part of the compositions according to the technical solution are suitable substances of natural plant origin or their mixtures soluble in water, such as ascorbic acid and its salts, various flavonoids, such as flavones, flavonols, flavanones, flavanonols, isoflavones, neoflavonoids, anthocyanidins, proanthocyanidins and their oligomers and polymers. From phytocomplexes obtained by extraction of plant materials containing substances of given category we may mention the extract from leaves of plant Camellia sinensis containing catechins from the group of flavanols, further the extract from Silybium marianum containing complex silymarin, extract from Curcuma longs containing complex curcumine, also extract from Rosmarinus officinalis containing rosemary acid, extract from Vitis vinifera containing proanthocyanidins and other polyphenolic substances of various type, extract from leaves of plant Ginkgo biloba containing flavonoids, fruits of Vaccinium myrtillus containing anthocyanidins, just like Euterpe oleracea. Apart from epicatechins contained in green tea, flavanol quercetin and polyphenol resveratrol are the most significant in common usage as part of food. Extract from French coastal pine tree Pinus pinaster ssp. atlantica contains a mixture of many bioflavonoids type catechins, oligomeric procyanidins and phenolic fruit acids with antioxidizing potential. Significant sources of ascorbic acid and its salts are extracts from plants Morinda citrifolia and Maipighia glabra. Concentration of phytocomplexas obtained by extraction from plants containing various antioxidants in the composition is in range from 0.05% by weight to 1.5% by weight, more preferably from 0.25% by weight to 1.5% by weight, most preferably from 0.50% by weight to 1.0% by weight. Water in the composition acts as a solvent for individual ingredients. Its ratio in the composition according to the technical solution of the invention is in range from 50% to 99%, more preferably from 65% to 90%, most preferably from 75% to 95%.

Ingredient used to adjust current acidity may be an acid or hydroxide, possibly amine or amino acid creating partial salts with nicotine or more advantageously buffer system acetate, citric, trolamin, tromethamol, carbonate, etc. Also suitable are dicarboxylic amino acids individual or in combination, such as glutamic acid or aspartic acid, or basic amino acids, such as lysine or arginine. With respect to nicotine stability and bioavailability it is advantageous to adjust pH value of solutions in range from 6.0 to 9.5, more preferably from 7.0 to 9.0, most preferably to pH 8.5.

Other ingredients, which can be advantageously exploited in the composition discussed by the invention, are preservation additives. As those suitable ones appear to be acids and salts of sorbic acid or benzoic acid. Other antimicrobic substances suitable for given purpose may include esters of parahydroxybenzoic acid, or parabens, especially methylparaben. Also suitable are various essentials oils, such as dove oil, thyme oil, wild thyme oil, eucalyptus oil, or extracts from these plants in semipolar solvents. Antimicrobial effect can be achieved also by propylene glycol or ethanol, which can be used also as extraction agents for plant materials. As preserving agents are suitable alcoholic or propylene glycol liquid extracts from plants, such as St. John's wort, Iceland lichen, Neem, sage, willow bark, etc.

Substances adjusting taste perceptions include mainly sweeteners. Most commonly used are various saccharides and inositols, such as glycerol, propylene glycol, saccharose, glucose, fructose, sorbitol, mannitol, xylitol, bee honey, etc. Significant group, which can be used for given purposes, is artificial non-nutritive sweeteners, such as saccharin salt, acesulfame salt, aspartame, neohesperidine dihydrochalcon, sucralose, extract from liquorice, extract from stevia, etc. Olfactory perceptions may be modified by essential oils, such as peppermint oil, spearmint oil, anise oil, fennel oil, eucalyptus oil, coriander oil, cumin oil, cinnamon oil, clove oil, thyme oil, wild thyme oil, ciropwort oil, juniper oil, marjoram oil, ginger oil, etc.

Compositions according to the invention may further also include other ingredients adjusting their physical parameters in a positive sense. Viscosity and surface tension of aqueous solutions of substances may be reduced by adding ethanol or propylene glycol, surfactants may also be used. As substances positively affecting conditions of surfaces in oral cavity, i.e. mucosae and teeth, may include wide range of vitamins and minerals, such the complex of vitamins B and their salts, vitamin C and its salts, esters and glycosides, vitamin H, coenzyme Q10, salts of zinc, copper, selenium, magnesium, calcium, manganese, etc. As antiinflammatory agents may be used alpha-bisabolol, allantoin, extract from St. John's wort, marigold, chamomile, etc.

Composition according to the invention is intended for application to oral cavity, onto the surface of buccal mucosa, gingival mucosa, hard palate and tongue by spray, i.e. by heterogenous or colloid dispersion of the composition in gas. As a generator of aerodispersion in the air may be either a pump or spraying may also be provided by flow of gas propeller from a pressure vessel. Propelling gas may advantageously be nitrogen or other chemically relatively inert gas, or mixture of alkanes, such as butane, isobutane or propane. Size of dispersed particles of mist or aerosol may be in range from 20 nm to 100 μm, preferably from 500 nm to 5 μm. For application is suitable, if the generator of aerodispersion is equipped with dispenser device. Volumes of liquid applied in single doses may be in range from 50 μl to 2000 μl, preferably from 150 μl to 500 μl. Listed parameters are indicative only, they are not a subject of the technical solution.

### EXAMPLES OF PREFERRED EMBODIMENTS

### Example 1, 2, 3:

| | Composition (% by weight) | | |
|---|---|---|---|
| Ingredient | Sample No. 1 | Sample No. 2 | Sample No. 3 |
| Nicotine | 1.00 | 2.00 | 3.00 |
| Citric acid | 0.95 | 1.55 | 2.10 |
| Acetylcysteine | 25.00 | 15.00 | 5.00 |
| Sodium Ascorbyl | — | 3.00 | 5.00 |
| Phosphate | | | |
| Potassium sorbate | 0.10 | 0.10 | 0.10 |
| Sodium benzoate | 0.10 | 0.10 | 0.10 |
| Sucralose | 0.07 | 0.08 | 0.10 |
| Glycerine | 5.00 | 10.00 | 15.00 |
| Grape seed extract | 1.50 | 0.75 | — |
| Perfume | 0.10 | 0.20 | 0.35 |
| Hydrogenated | 0.12 | 0.12 | 0.12 |
| pegylated castor oil | | | |
| Sodium hydroxide | q.s. pH 7.0-9.0 | q.s. pH 7.0-9.0 | q.s. pH 7.0-9.0 |
| Water | up to 100.00 g | up to 100.00 g | up to 100.00 g |

### Example 4, 5, 6, 7:

| | Composition (% by weight) | | | |
|---|---|---|---|---|
| Ingredient | Sample No. 4 | Sample No. 5 | Sample No. 6 | Sample No. 7 |
| Nicotine | 1.00 | 0.2 | 2 | 1 |
| Citric acid | 0.95 | 1.55 | 1.55 | 2.10 |
| Primula veris extract | 2.00 | — | — | — |
| Hedera helix extract | — | 3.00 | — | — |
| Liquiritia glabra extract | — | — | 10.00 | — |
| Tussilago farfara extract | — | — | — | 1.00 |
| Camellia sinensis | 1.00 | — | — | — |
| extract | | | | |
| Rosmarinus officinalis | — | 0.50 | — | — |
| extract | | | | |
| Equisetum arvense | — | — | 0.30 | — |
| extract | | | | |
| Ginkgo biloba extract | — | — | 1.00 | — |
| Vitis vinifera extract | — | — | — | 0.10 |
| Potassium sorbate | 0.10 | 0.10 | 0.1 | 0.10 |
| Sodium benzoate | 0.10 | 0.10 | 0.1 | 0.10 |
| Sucralose | 0.07 | 0.08 | 0.07 | 0.10 |
| Glycerine | 5.00 | 10.00 | 10 | 15.00 |
| Aroma | 0.10 | 0.20 | 0.3 | 0.35 |
| Hydrogenated pegylated | 0.12 | 0.12 | 0.12 | 0.12 |
| castor oil | | | | |
| Sodium hydroxide | q.s. pH 7.0-9.0 | q.s. pH 7.0-9.0 | q.s. pH 7.0-9.0 | q.s. pH 7.0-9.0 |
| Water | up to 100.00 g | up to 100.00 g | up to 100.00 g | up to 100.00 g |

Preparation procedure: Individual ingredients, gradually added, are dissolved in water. Solution is filtered if necessary and filled into pressure vessels with dispenser valve or into closed containers with dispenser valve driven by manually operated pump.

### INDUSTRIAL APPLICABILITY

Composition according to the invention may be produced by pharmaceutical industry. Preparation is suitable for transmucosal absorption of nicotine for the purpose of smoking cessation.

1. A method for buccal absorption of nicotine into a human's system circulation and distribution to the person's central nervous system for the purpose of smoking cessation or nicotine substitution, comprising
administering via buccal absorption, in a form of colloid or heterogene aerodispersion by spray device a composition the composition comprising
a nicotine solution in a form of a base and/or its salt with an organic acid in concentration 0.01 to 8.00% by weight,
substances with mucolytic effect selected from the group formed by acetylcysteine in an amount of 3.0 to 30.0% by weight and/or phytocomplexes with mucolytic effect obtained by extraction from plants selected from the group Primula veris, Primula elatior, Hedera helix, Eucalyptus globulus or Liquiritia glabra in a concentration from 0.50 to 15.00% by weight, 0.05 to 1.50% by weight of phytocomplexes with antioxidizing effect selected from the group of flavones, flavonols, flavanones, flavanonols, isoflavones, neoflavonoids, anthocyanidins, proanthocyanidins and their oligomers and polymers obtained by extraction from plants Camellia sinensis, Curcuma longs, Rosmarinus officinalis, Sylibium marianum, Vitis vinifera, Ginkgo biloba, Euterpe oleracea, Vaccinium myrtillus, Merinda citrifolia, Malpighia glabra, and
a buffer or a substance adjusting current acidity in the range of pH value from 7.0 to 9.0.

2. The method according to claim 1, wherein the composition, as a solvent contains water in concentration 50 to 99% by weight or its mixture with polyhydric alcohol, such as glycerol, propylene glycol, and/or saccharide selected from the group mannitol, xylitol, glucose, fructose, saccharose in a concentration from 2 to 50% by weight.

3. The method according to claim 2, comprising at least one component from the group of substances adjusting taste perceptions, substances adjusting olfactory perceptions, antimicrobials, sequestrants, antioxidants, solubilizing agents and agents facilitating wettability, substances improving physical parameters of applied compositions, such as viscosity and surface tension, and substances improving condition of oral cavity surfaces, i.e. mucosae or teeth.

4. The method according to claim 3, wherein a single application dosage contains 0.05 to 3.00 mg of nicotine.

5. The method according to claim 4, wherein a single application dosage contains 0.10 mg to 2.00 mg of nicotine.

6. The method according to claim 5, wherein a single application dosage contains 0.25 mg to 1.00 nag of nicotine.

7. The method according to claim 2, wherein a single application dosage contains 0.05 to 3.00 mg of nicotine.

8. The method according to claim 7, wherein a single application dosage contains 0.10 mg to 2.00 mg of nicotine.

9. The method according to claim 8, wherein a single application dosage contains 0.25 mg to 1.00 mg of nicotine.

10. The method according to claim 1, comprising at least one component from the group of substances adjusting taste perceptions, substances adjusting olfactory perceptions, antimicrobials, sequestrants, antioxidants, solubilizing agents and agents facilitating wettability, substances improving physical parameters of applied compositions, such as viscosity and surface tension, and substances improving condition of oral cavity surfaces, i.e. mucosae or teeth.

11. The method according to claim 10, wherein a single application dosage contains 0.05 to 3.00 mg of nicotine.

12. The method according to claim 11, wherein a single application dosage contains 0.10 mg to 2.00 mg of nicotine.

13. The method according to claim 12, wherein a single application dosage contains 0.25 mg to 1.00 mg of nicotine.

14. The method according to claim 1, wherein a single application dosage contains 0.05 to 3.00 mg of nicotine.

15. The method according to claim 14, wherein a single application dosage contains 0.10 mg to 2.00 mg of nicotine.

16. The method according to claim 15, wherein a single application dosage contains 0.25 mg to 1.00 mg of nicotine.

17. The method according to claim 1, wherein the buffer or substance adjusting current acidity pH value is about 8.5.

18. The method according to claim 1, wherein the nicotine solution is in the form of the base and/or its salt with the organic acid in a concentration of 0.10 to 4.00% by weight.

19. The method according to claim 1, wherein the nicotine solution is in the form of the base and/or its salt with the organic acid in a concentration of 0.20 to 1.00% by weight.
